(19) United States
(12) Patent Application Publication
(10) Pub. No.: US 2026/0144950 A1
(43) Pub. Date: May 28, 2026

(54) EXPANDABLE ENDOTRACHEAL TUBE AND METHOD FOR USE OF SAME

(71) Applicant: Mary Hitchcock Memorial Hospital, for itself and on behalf of Dartmouth-Hitchcock Clinic, Lebanon, NH (US)

(72) Inventors: Alexander T. Abess, Hanover, NH (US); Carolina Almonte, St. Cloud, FL (US); Lindsey E. Beaudoin, Bedford, NH (US); Alessandra Cristine Bryan, Quechee, VT (US); Yvon Fleissner Bryan, Quechee, VT (US); Isaac Mitchell Hanover, Cockeysville, MD (US); Nolan Baine Sankey, Steamboat Springs, CO (US)

(21) Appl. No.: 19/121,981

(22) PCT Filed: Oct. 17, 2023
(86) PCT No.: PCT/US2023/035341

§ 371 Date: Apr. 17, 2025

Related U.S. Application Data
(60) Provisional application No. 63/416,917, filed on Oct. 17, 2022.

(51) Int. Cl. A61M 16/04; A61B 1/267

(52) U.S. Cl. CPC A61M 16/0402; A61M 16/044; A61M 16/0463; A61B 1/2676

(57) ABSTRACT

This invention provides an expandable endotracheal tube (ETT) and method for use of the same, including a frame having a plurality of hoops joined by longitudinal ribs, the hoops formed into a compressed configuration with torsional spring force imparted thereby, a collapsible shell covering the frame that defines a sealed tube between a proximal end and a distal end, and a retaining structure that maintains the hoops in the compressed configuration with the frame and the shell defining a compressed outer diameter. The retaining structure is selectively releasable to cause to the frame and the shell to expand to an expanded diameter that is greater than the compressed diameter, and/or can include a member that passes through a plurality of holes located along overlapping circumferential positions of the frame and the shell folded therearound. The retaining structure can comprise a string under tension.

### RELATED APPLICATION

This application claims the benefit of U.S. Provisional Application Ser. No. 63/416,917, entitled EXPANDABLE ENDOTRACHEAL TUBE AND METHOD FOR USE OF SAME, filed Oct. 17, 2023, the teachings of which are expressly incorporated herein by reference.

### FIELD OF THE INVENTION

This invention relates to endotracheal tubes, and more particularly to endotracheal tubes with an expandable geometry.

### BACKGROUND OF THE INVENTION

Fiber optic intubation is a well-known treatment regimen for management of (e.g.) difficult airways in the perioperative, emergency, and critical care environments. The term, “difficult airway” is commonly employed by those of skill in the art, and represents patients whose anatomy or clinical situation make traditional methods of intubation challenging and/or especially dangerous. Fiber optic intubation is a procedure when a flexible bronchoscope is used to enter the patient's trachea via the nose or mouth. The flexible bronchoscope then serves as a conduit over which a traditional endotracheal tube is advanced into the patient's trachea.

Current endotracheal tubes (ETTs) are typically constructed from a biocompatible polymer, such as semi-rigid PVC, and define an outer diameter of approximately 10 mm. These endotracheal tubes are notably larger than flexible bronchoscopes. As such, although it can be relatively straightforward to maneuver the flexible bronchoscope into a patient's trachea, advancement of the endotracheal tube itself can be complicated by tissue trauma (causing bleeding and increased difficulty of visualization and placement), as well as pain/discomfort to the patient because of its outer diameter. In addition, the size of the outer diameter of the endotracheal tube sometimes is too large to even bypass whatever anatomy (e.g. a tumor) is responsible for creating the difficult airway condition.

More, generally, about 50 million intubations are performed globally each year, and virtually all of them rely on the use of state-of-the-art ETTs. The ongoing COVID-19 pandemic has increased the demand for intubations. Thus, airway management is increasingly crucial now than ever, yet the general procedure and associated intubation device(s) used has/have remained stationary for over many years. Intubation procedures must be carried out in such a way that results in more benefit than detriment to the patient—however, the large diameter size of the state-of-the-art ETTs often results in complications and or challenges in placement. That is, the size of a typical ETT can cause significant pain or discomfort for the patient (even in cases of a non-difficult airway) during insertion, and in some instances, may damage to the airway, which requires the need for general anesthesia. Those familiar with the art will recognize the desire for more straightforward intubation process that can be less traumatic to the patient.

FIG. 1 shows a typical ETT 100 according to a prior art implementation. The tube body 110 is constructed from (e.g.) biocompatible PVC. The distal tip 112 includes a bevel to assist in insertion, and includes a “Murphy Eye” (additional lumen) on the side of the ETT adapted to avoid airway obstruction. Adjacent the distal end is an inflatable cuff 115 that seal the connection between the ETT and walls of the trachea to prevent a leak from the lungs. An inflation lumen 116 is disposed along the body 110 and joins to an external inflation tube 120 connected to a pilot balloon 122 that enables the user to inflate the cuff using (e.g.) a syringe filled with air (or possibly saline) that passes through a check valve 124. The body can include indicia 130 and/or radioluminescent marker(s), as shown, to assist the user in determining insertion depth into the patient's airway. A 14-15 mm adaptor 140 is provided at the proximal end for interconnection with respiratory equipment and other instrumentation.

Those familiar in the art will recognize that direct laryngoscopy is the most common means of visualizing the glottic opening for placement of an endotracheal tube. Video laryngoscopy is also widely used, as is flexible bronchoscopy for placement of endotracheal tubes. Although these three approaches use different mechanisms of visualizing the glottic opening, they are similar in that they (1) require visualization of the glottic opening into the trachea and (2) occasionally create circumstances when the glottic opening can be visualized, but a standard diameter endotracheal tube cannot be easily advanced. FIG. 2, thus, shows an exemplary ETT 210 according to a prior art implementation, in which a flexible fiber optic bronchoscope 220 has been removably attached to the proximal end 222 of the ETT 210 so that the fiber 230 (shown in phantom) passes through the lumen of the ETT with the distal end 232 of the fiber exposed at the corresponding distal end 224 of the tube 210. The fiber distal end 232 includes appropriate optics for imaging the patient's internal anatomy on an interconnected display 240. The flexible bronchoscope 220 and associated fiber 230 is withdrawn (arrow 250) from the lumen after successful placement in the patient.

While it may be possible to employ a smaller outer diameter ETT to ease the insertion procedure, physics and breathing mechanics would render such a small diameter tube body suboptimal for respiration due to air flow resistance. Flow resistance is inversely proportional to the 4th power of the airway radius. Thus, even small changes in endotracheal tube diameter become significant for breathing mechanics.

It is desirable to provide a safer intubation procedure that is less complex for clinicians that reduces the need for general anesthesia. While other medical disciplines, such as cardiology, in which devices like stents, artificial valves, etc. (and the techniques surrounding them) have advanced dramatically, have seen dramatic advancements over the last 20 years in their underlying technology, ETTs used for airway management have remained relatively the same. It is desired to provide an ETT that can be inserted at a reduced outer diameter size (e.g.) and can be employed to deliver air to the patient at a standard working diameter size (e.g. 10 mm) once placed in the patient's airway. Such size reduction prior to, and/or during, insertion would desirably allow clinicians to use only minimal sedation, and/or apply topical anesthesia to insert the ETT, and no need for general anesthesia. The result would be fewer procedural challenges and better patient outcomes.

### SUMMARY OF THE INVENTION

This invention overcomes the prior art by providing a flexible and expandable endotracheal tube (ETT) which makes placement of breathing tubes easier and safer, The expandable ETT herein enables a step change in size from an outer diameter of approximately 5 mm during delivery/insertion to an expanded size of 10 mm. The collapsed/compressed configuration of the ETT can define at least a 2.5 mm inner lumen through which a flexible bronchoscope (or other tracheal navigation technique/device that should be clear to those of skill) can pass. The inner diameter ETT in its expanded form can be between 8 mm and 10 mm in an example, and/or other size ranges—for example expanding to 12-13 mm, or larger, where appropriate—for example for prolonged use, such as where a patient resides in an intensive care (ICU) environment and/or requires long-term intubation. The expandable frame in this embodiment underlies a flexible, collapsible shell. The frame comprises a series of a bent hoops joined by longitudinal ribs, and employs a torsional spring technique which meets necessary performance criteria for expansion while resisting collapse after expansion. The construction of the expandable ETT is also relatively cost effective using existing manufacturing techniques and materials.

In an illustrative embodiment, an expandable endotracheal tube and method for use of the same comprises an expandable endotracheal tube (ETT) having a frame having a plurality of hoops joined by longitudinal ribs, the hoops formed into a compressed configuration with torsional spring force imparted thereby. A collapsible shell covers the frame that defines a sealed tube between a proximal end and a distal end. A retaining structure maintains the hoops in the compressed configuration with the frame and the shell defining a compressed outer diameter. The retaining structure can be selectively releasable to cause to the frame and the shell to expand to an expanded diameter that is greater than the compressed diameter. Illustratively, the retaining structure can comprise a member that passes through a plurality of holes located along overlapping circumferential positions of the frame, with the shell folded therearound. The retaining structure can comprise a string under tension. The string can pass through holes in each of the ribs when adjacent to each other, with the ribs moving circumferentially to circumferentially spaced-apart positions in the expanded configuration. The frame can further include a stop that resists compression after the frame expands to the expanded configuration. In various embodiments, a cuff is provided adjacent to the distal end, which collapses in the compression. The cuff can be interconnected to an inflation source having a conduit that extends along a longitudinal length of the frame and shell. In other embodiments, at least a portion of the frame and the shell, adjacent to a distal end of the ETT defines, when in the expanded diameter, an integral cuff, adjacent to the distal end with a larger diameter than the adjacent shell at the expanded diameter, so as to define a seal with the airway. In various embodiments, a compression mechanism can be arranged to allow for returning the frame to a diameter that is smaller than the expanded diameter to facilitate removal from the airway. Illustratively, the frame can define an inner lumen in the compressed configuration adapted to slidably receive a flexible bronchoscope, or other (e.g. tracheal) delivery mechanism/device. In various embodiments, the frame and shell can be constructed and arranged to expand from a compressed outer diameter of approximately 5 mm to an expanded outer diameter of between approximately 8 mm and 10 mm. In other embodiments, the frame and shell can be constructed and arranged to expand from a compressed outer diameter of between approximately 5 mm and 8 mm (and more particularly, 7 mm-8 mm) to an expanded outer diameter of between approximately 12 mm and 13 mm. In various embodiments herein, the proximal end of the ETT includes a fitting for connection to respiratory equipment. Illustratively, the retaining structure that selectively maintains the compressed configuration can comprise a semi-rigid rod.

In another illustrative embodiment, the above described ETT can be provided with another frame having a plurality of hoops joined by longitudinal ribs, the hoops formed into a compressed configuration with torsional spring force imparted thereby, and another collapsible shell that covers the frame, and that defines a sealed tube between a proximal end and a distal end. The same, or a separate, retaining structure can causes the other frame and the other shell to define a compressed outer diameter, and can be releasable to cause to the frame and the shell to expand to an expanded diameter that is greater than the compressed diameter. A lumen can be provided along the ETT body for inflating the cuff to the large diameter based upon a proximally located inflation mechanism. Both the shell, and the other shell, can be maintained adjacent to each other along at least a portion of respective lengths thereof—so as to define a double lumen ETT.

In another illustrative embodiment, the above-described ETT can be used (in a treatment method) by associating an inner lumen of the ETT, in the compressed configuration, with a tracheal delivery device. The ETT is then guided into an airway of the patient using information from the delivery device. The delivery device is subsequently disengaged, and the retaining structure is actuated to place the ETT into the expanded configuration. The delivery device can be (among other mechanisms and/or associated techniques) a flexible bronchoscope that is selectively inserted within the lumen in the compressed configuration, and withdrawn therefrom, when guidance is completed. The method can then include inflating a cuff adjacent to the distal end of the ETT to seal the airway.

### BRIEF DESCRIPTION OF THE DRAWINGS

The invention description below refers to the accompanying drawings, of which:

FIG. 1 is a diagram of a typical endotracheal tube adapted for fiber optic guidance with a flexible bronchoscope, and placement, according to the prior art;

FIG. 2 is a diagram showing an exemplary ETT with a flexible bronchoscope attached thereto according to a prior art implementation;

FIG. 3 is a perspective view of the frame of an expandable ETT according to an illustrative embodiment, shown in a compressed configuration with a release string engaged to secure the structure;

FIG. 4 is a perspective view of the frame of FIG. 3, shown in an expanded configuration with the release string disengaged;

FIG. 5 is a perspective view of the ETT according to an illustrative embodiment, showing the frame of FIG. 3 is the compressed configuration with an associated, flexible outer shell in a folded state for insertion into a patient's airway;

FIG. 6 is a diagram showing a side view of the ETT according to an illustrative embodiment including the shell and an inflatable cuff with inflation bulb connected by a conduit;

FIG. 7 is a fragmentary diagram of the components of the release mechanism for the frame of FIG. 3;

FIG. 8 is a fragmentary perspective view of the frame shown in a flattened orientation before assembly into the ETT structure;

FIG. 9 is a fragmentary plan view of the flattened frame of FIG. 8;

FIG. 10 is a side view of the flattened frame of FIG. 8;

FIG. 11 is a somewhat schematic, fragmentary perspective view of a string-based release mechanism for the ETT according to an illustrative embodiment;

FIG. 12 is a somewhat schematic, fragmentary perspective view of a flexible rod-based release mechanism for the ETT according to an alternate embodiment;

FIG. 13 is a perspective view of relative forces exerted on the ETT during insertion into a patient's airway;

FIG. 14 is a perspective view of generalized radial force balance for the ETT when inserted into a patient's airway in a resting state;

FIG. 15 is a cross-sectional view of the ETT, including frame, shell and cuff showing relative radial forces during air inspiration by the patient;

FIG. 16 is an exposed side view of an ETT with a frame-formed, increased-diameter cuff segment along a longitudinal axis thereof, according to an alternate embodiment,

FIG. 17 is a perspective view of a full-length version of the ETT according to an illustrative embodiment in the compressed configuration;

FIG. 18 is a perspective view of the ETT of FIG. 17 is the expanded configuration;

FIG. 19 is a cross section of the ETT in the compressed configuration, taken along line 19-19 of FIG. 17; and

FIG. 20 is a cross section of the ETT in the compressed configuration, taken along line 20-20 of FIG. 19.

### DETAILED DESCRIPTION

### I. Structural Overview

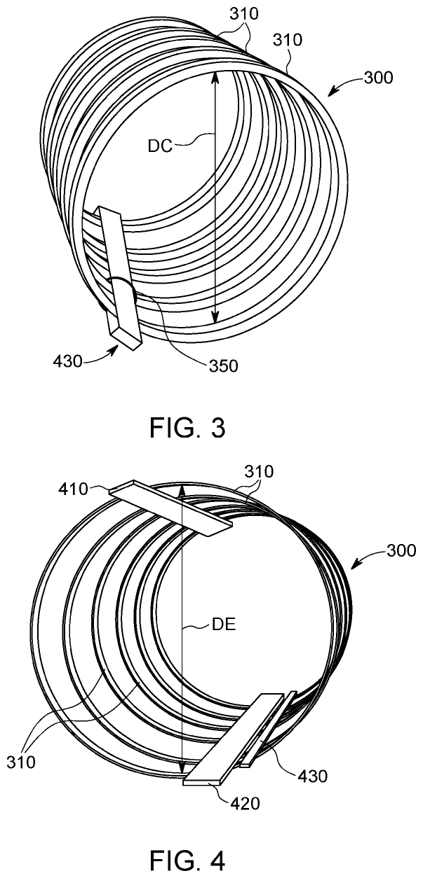

FIGS. 3 and 4 show a frame assembly 300 that is adapted to enable an expandable ETT according to an illustrative embodiment. The frame assembly (or “frame”) 300 operates on the principal of a torsion spring. It includes a set of hoops 310 that are formed, under elastic bending stress, into a series of hoops disposed along an axis of elongation (the longitudinal axis). The frame 300 in FIG. 3 is shown with the diameter DC of the hoops reduce to an approximate minimum, compressed diameter that, in operation can be less than 5 mm. Conversely, in FIG. 4 the frame 300 is shown radially expanded under spring bias (bending-induced hoop stress) of the hoops 310 to an approximate maximum diameter DE of slightly less than 10 mm.

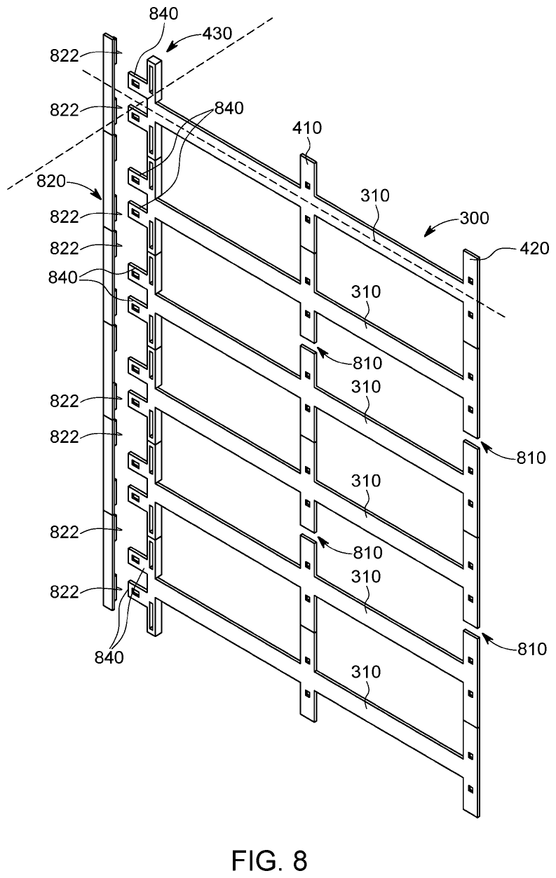

As shown clearly in FIG. 4, the hoops 310 are joined together longitudinally by a pair of diametrically opposed ribs 410 and 420 that are part of a unitary structure (See FIGS. 8-10, described below) that can be constructed by a variety of manufacturing techniques including molding, 3D printing, stamping/die-cutting polymer sheet, etc. The material used to form the frame is highly variable. For example, a biocompatible PLA, PET, etc. can be used. As also described below, a separate longitudinal spine member that, in this embodiment, can be constructed from the same, or different, polymer relative to the frame hoops 310 and ribs 410, 420. It is either friction-fit or adhered an underlying base rib as described further below. The spine serves as a stiffener for the overall tube structure in the expanded configuration and can also serve as a stop to resist re-compression of the ribs 310 once deployed into the expanded configuration.

Deployment of the ribs from the compressed into the expanded configuration is controlled by a release string 350 in this embodiment. The release string passes through holes in the ribs and hoops (described further below) in a manner that ties the ribs and hoops in an overlapped arrangement to maintain a compressed configuration (FIG. 3). The release string 350 is secured by a knot or other fixation structure so that it resists pullout from the frame holes in the compressed configuration. The user can deploy the frame (under its inherent hoop stress) into the expanded configuration (FIG. 4) by cutting, or otherwise releasing holding tension in, the string 350 after the structure is inserted into a patient's airway. In other embodiments, the string or other ligature can be a sufficient length to allow it to be paid out to, thereby, expand the structure, and when the task is completed, allow the string to be tensioned/pulled proximally to re-compress the structure for ease of withdrawal/removal from the patient.

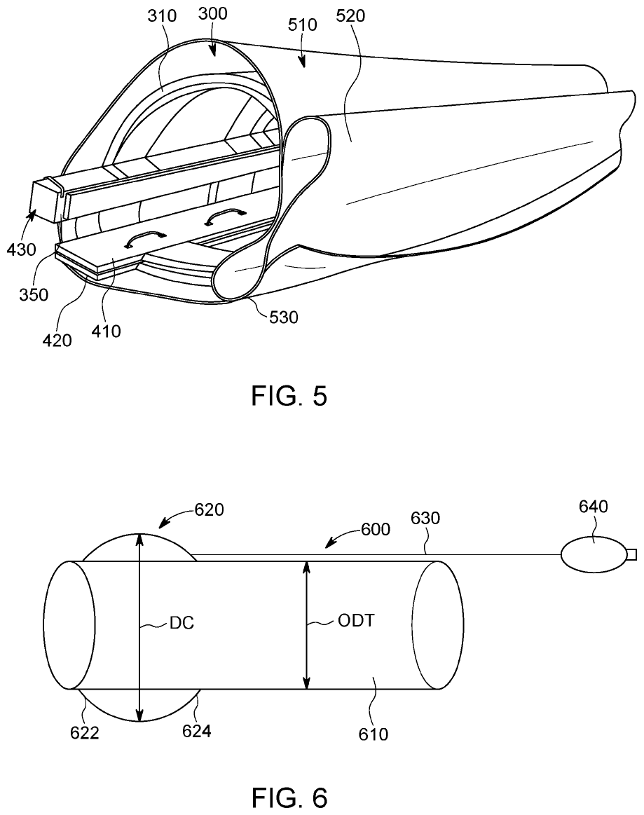

The exterior of the frame 300 is covered with a sealed, thin-wall shell 510 as shown in FIG. 5. The shell 510 can be constructed from any acceptable, biocompatible material. In general, it defines a continuous, seamless tube for (e.g.) by molding, extrusion, etc., with an expanded outer diameter that is no more than approximately 10 mm. The shell material can be PVC, PET, or any other appropriate, biocompatible/medical grade, stretchable thin-wall material. Such material can be formed from a sheet that is cut to size and welded or adhered along a seam to form a tube; or formed as a seamless tube using, for example, blow molding, extrusion or other appropriate manufacturing techniques clear to those of skill. As shown particularly in FIG. 5, the shell 510 defines a series of longitudinal folds 520 and 530 that accommodate the difference in perimeter when the frame 300 is compressed. The shell folds 520, 530 are fully taken up by the expanded diameter (DE in FIG. 4) of the frame 300 when the structure is deployed after insertion into a patient's airway. Note that the particular arrangement of folds depicted is only exemplary of a wide range of compacting geometries for the shell in the compressed configuration. In alternate arrangements, the shell and be folded more evenly about the frame perimeter and/or can be folded inwardly to avoid snagging on the airway during insertion. Depending upon the material, the shell can define an elastically deformable set of folds that maintain a close-conforming fit to the compressed frame about the perimeter in the manner of an accordion. The spring force of these accordion folds is overcome by the frame's deployment force, and such folds become substantially flattened into a cylindrical tube wall. Other folding geometries should be clear to those of skill. Such geometries can include a small amount of elastic flexure as the frame expands fully, thereby forming a taut tubular structure. Note that the maximum range of expansion for the ETT herein can be varied. In an embodiments, the expandable nature of the structure can allow for a size that is desirably larger in certain embodiments—for example 12-13 mm. In such embodiments, the use of a mechanism (e.g. a proximally withdrawn pull string) that allows for at least partial recompression of the structure prior to withdrawal from the patient can be desirable.

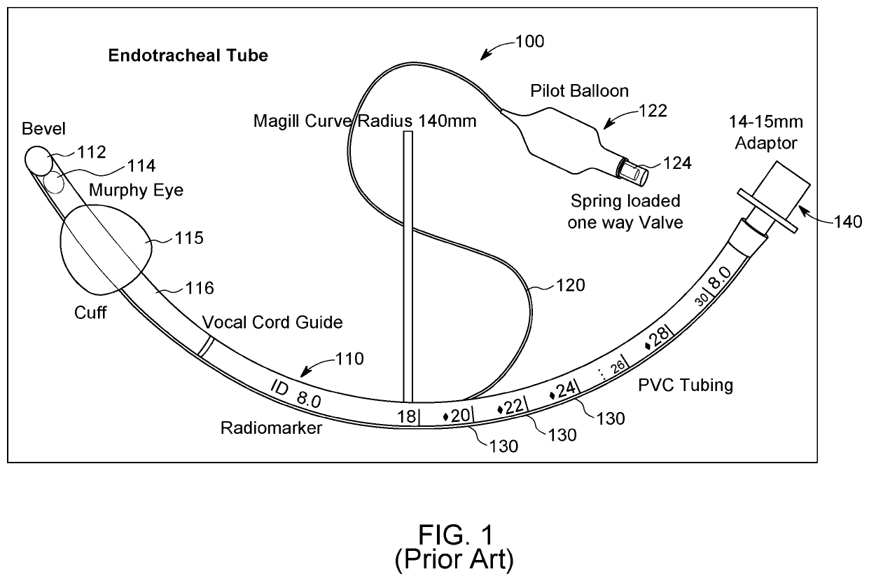

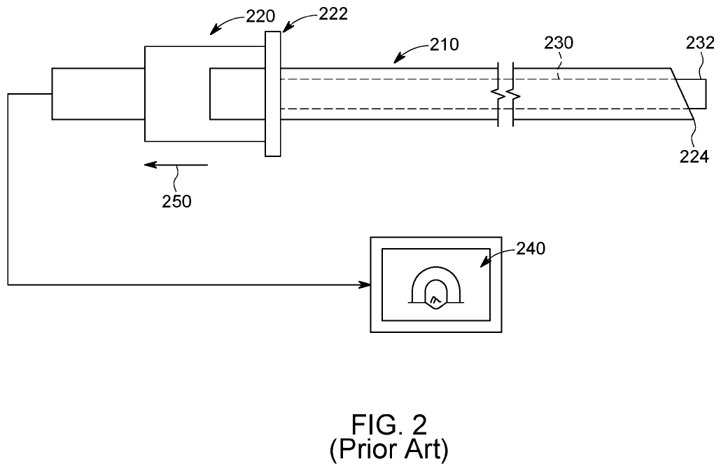

FIG. 6 depicts a schematic version of the expanded ETT 600 according to an illustrative embodiment. The expanded shell 610 includes, at a distal end thereof, an inflatable cuff 620. The cuff 620 can be constructed from any acceptable, somewhat elastic, biocompatible material—for example PVC or PET. It can be generally similar in material and construction to that of the conventional ETT described above (FIGS. 1 and 2). It should be capable of folding, along with the shell 610 so as to attain a compressed diameter for insertion into the airway. In an embodiment, the cuff 620 is adhered in sealed manner to the surface of the shell 610 about a confronting perimeter at its distal edge 622 and proximal edge 624. The cuff 620 remains unsealed from the shell 610 in its middle 626, so as to allow a relative increase in cuff diameter DC beyond the outer diameter ODT of the ETT body shell 610 when inflated. An inflation line 630 can be provided on, beneath or within the surface of the shell, or another location that runs longitudinally along the tube to an external location. The line 630 interconnects the cuff 620 with and external inflation balloon 640, which can be of conventional or custom design.

The inner lumen of the ETT can be of sufficient diameter in the compressed configuration to (optionally) allow for threading of a guiding fiber optic therein. This enables visual guidance of the ETT into a patient's airway using a flexible bronchoscope. Also, while not shown, the distal and/or proximal ends of the expandable ETT herein can be constructed similarly to a conventional, non-expandable ETT, such as that shown in FIGS. 1 and 2. More particularly, the frame can be constructed with a bevel and/or include other end fittings that perform a desired task (e.g. airway access, interconnection with respiratory mechanisms, etc.) while also facilitating insertion of the ETT structure while in a compressed state. Note, while it is contemplated that the embodiments herein can be adapted to receive a flexible bronchoscope to aid in guidance, it is expressly contemplated that the ETT lumen in various embodiments can be sized smaller that the outer diameter of a typical bronchoscope, or otherwise not contemplate the use of one, in a non-visual delivery technique—which is facilitated by the novel aspects of the ETT herein.

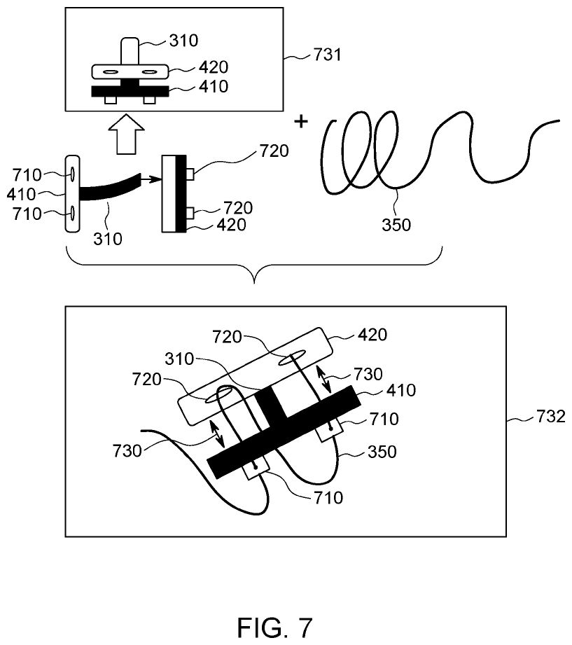

With reference to FIG. 7, the structure and function of the retention mechanism is described in further detail. As shown, the hoop 310 and interconnected rib 410 are adapted to compress into a form (window 731) that confronts and overlaps the adjacent rib 420 under spring tension induced by the hoops. The tension (arrows 730) is retained by the string 350, which is threaded (window 732) through opposing holes 710 and 720 in respective ribs 410 and 420. Hence the string 350 is secured in a taut state when the ribs confront and the associated frame is fully compressed to a minimum diameter. Cutting or otherwise releasing holding tension in the string results in release of stress in the frame hoops, which results in expansion to the maximum diameter. Note that the outer shell (510 in FIG. 5) restricts further outer expansion of the frame as the hoops are restrained by the cylindrical shape of the shell's inner wall.

With reference to FIGS. 8-10, an exemplary arrangement of the frame structure 300 in an unassembled state. The frame 300 in this embodiment is formed as a flat, unitary element from an appropriate, elastically deformable and biocompatible polymer. It can be formed by cutting/stamping sheet material, molding, 3D printing, or any other acceptable formation technique. In manufacturing, the frame 300 is, thus, adapted to be rolled into a cylinder from the depicted flat shape and covered with the outer shell. In alternate embodiments, the frame can be formed as a pre-rolled, cylindrical unit with a diameter larger than the maximum expanded diameter so as to induce spring force in the structure. Likewise, it is contemplated that various techniques for co-molding (co-forming) the frame and shell can be employed within ordinary skill in the art.

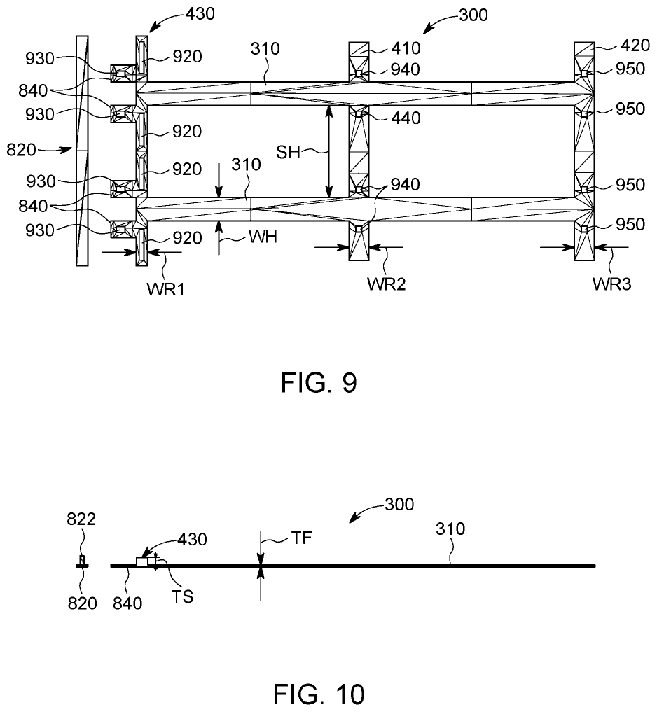

As shown, the ribs 410 and 420 include optional breaks 810 along their longitudinal length. This assist with flexibility of the ETT in both and expanded and compressed state. This can also allow a frame of a desired length to be constructed from segments of ribs and hoops. The frame structure further includes a spine rib 430 and a confronting spine element 820 that is adapted to overlie the spine rib 430 in an assembled state. A series of interlocking tabs 822 and slots 920 on the spine element 820 and rib 430 facilitate retention of the element 820 within the frame. The element can act, in part as a stop that prevents recompression of the frame once deployed. Alternate stops, or hoop stress exclusively, can also be used to resist recompression of the deployed/expanded frame once inserted in a patient. The spine rib 430 includes a set of terminal end extensions 840 relative to each hoop 310. There are holes 930 (FIG. 9) in the extensions 840 that receive the retaining string in the compressed state. The string likewise passes through adjacent holes 940 and 950 in ribs 410 and 420 respectively. Note that in alternate embodiments the location and placement of holes can be varied from the depicted arrangement. Holes 930, 940, 950 on different ribs/extensions can overlap, and/or be longitudinally offset relative to each other.

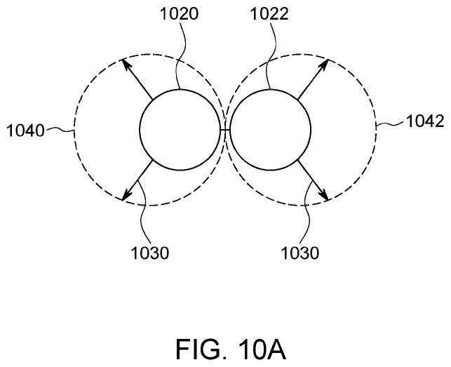

The thickness TF (FIG. 10) of some or all of the frame 300 can be highly variable, both overall, and at various locations. For example, the spine rib thickness TS is greater than that of the other ribs 410, 420 and hoops 310. Likewise, the width WR1, WR2 and WR3 (FIG. 9) of each respective rib 430, 410 and 420 can vary. The thickness and width is chosen based upon mechanical performance analysis and test results, and is designed to ensure that the structure performs as desired. The dimensions needed for optimization should be clear to those of skill. In general, the thickness TF is desirably sized so that the outer diameter of the ETT is reduced to approximately 5 mm in the compressed configuration, while the inner diameter in this configuration is provides an unobstructed inner diameter of at least 2.5 mm. Hence, thickness TF is typically no greater than 1.25 mm and typically less. Similarly, the spacing SH of hoops 310 along ribs, and their width WH, should be chosen to balance ETT flexibility with resistance to collapse and/or kinking during, for example, air inspiration by the patient.

It is further contemplated that the ETT herein can be constructed in a manner that defines a double lumen endotracheal tube. Those of skill can recognize that such is a specific endotracheal tube used for individual lung isolation. By way of non-limiting example, one of skill can implement a double lumen ETT based upon the teachings herein using the contemplated expansion mechanism in two adjacent expandable ETTs that are affixed to each other and allow placement of one distal lumen in a right or left mainstream bronchus and the other distal lumen is either in the opposing right or left bronchus (respectively), or in the main tracheal lumen. A schematic diagram of such a structure is shown in cross section in FIG. 10A. The two side-by-side lumens 1020 and 1022 are adapted to move (arrows 1020) from a compressed arrangement to an expanded arrangement 1040, 1042, respectively once paced in the appropriate locations in the patient's anatomy. The expansion of the two lumens can be implemented using a pair of side-by side expandable rib structures as described herein with overlying shells that are folded, or otherwise compacted, into the smaller compressed diameter. The side-by-side shells covering the rib structures can be joined together along a portion of their length, and typically, unjoined at the distal end for placement in different locations. The shells maintain separate air circuits along their entire length, or at least at the unjoined distal end thereof. A separate overlying shell can be provided over the two lumens along a portion thereof in certain embodiments. This overlying shell would expand in response to expansion of the two underlying lumens.

### II. Alternate Retention Structures

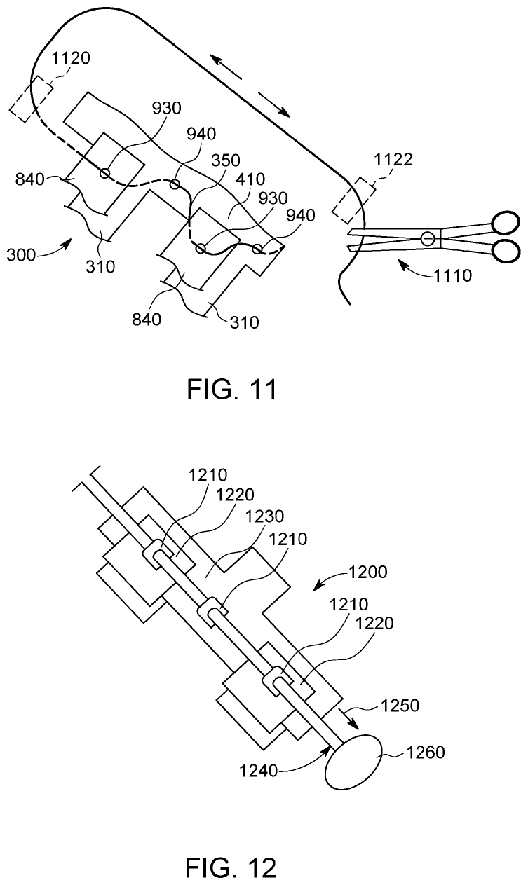

Reference is made to FIGS. 11 and 12 which show two alternate structures and associated techniques for retaining the frame in a compressed state. FIG. 11 shows the retention string 350, described above passed through holes 930 and 940. The string 350 in this embodiment forms a continuous loop under tension that can be released by cutting (exemplary scissor 1110). The string 350 can then be pulled out or allowed to retract into the expended structure. Alternatively, the string can be fixed at the distal end and/or include a stopper 1120 (shown in phantom). Tension is maintained by opposing, proximal stopper 1122. Collectively, the two stoppers (or other fixation mechanisms) maintain a tensioned string with the frame fully compressed. By cutting or releasing the proximal stopper 1122, the string withdraws from the holes 930, 940 as the frame expands.

As shown in FIG. 12, the frame 1200, in an alternate embodiment, can include a set of loops or guides 1210 on confronting frame surfaces 1220 and 1230 that receive a semi-rigid retaining rod 1240. The rod is constructed from biocompatible metal or polymer of appropriate diameter to resist plastic deformation and resist the expansion spring force of the frame 1200. When withdrawn, following placement of the ETT in the patient's airway, the rod 1240 is withdrawn proximally (arrow 1250), allowing the frame to deploy into the expanded configuration. The rod 1240 can include a tab or other graspable (e.g. by fingers, tweezers, forceps, etc.) structure 1260 at its proximal end to facilitate withdrawal.

### III. Force Balance

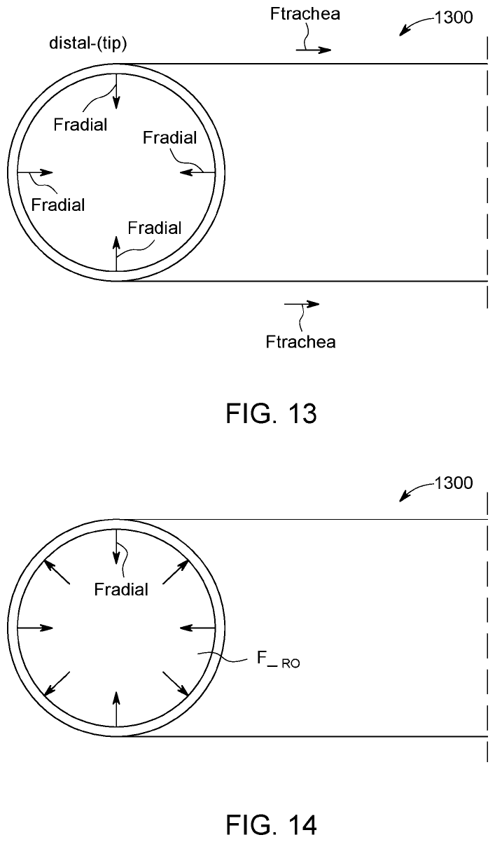

As described above, the expandable ETT contemplated herein should resist collapse once expanded. A variety of inwardly and outwardly directed radial forces (as well as axial/longitudinal forces) are imparted on the structure at various times during its insertion, expansion and operation. As shown in FIG. 13, the patient's airway (trachea) exerts a radially inwardly directed force, F_{radial} during insertion on the body of the ETT 1300. The trachea, itself also resists the act of longitudinal insertion in the distal direction with a proximally directed force, F_{trachea}. Both of which forces are overcome by the structure of the compressed ETT 1300. Upon expansion, as shown in FIG. 14, the force, F_{radial} is overcome, and balanced by an outward radial force, F__{RO} that is generated by the spring force of the frame and maintained (optionally) in part by a retaining structure that engages the expanded frame of the ETT 1300.

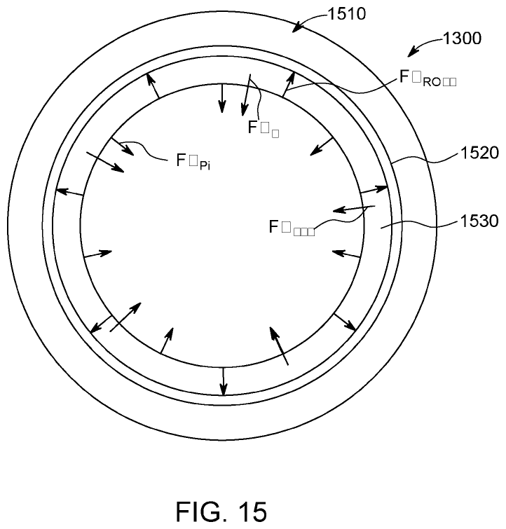

More particularly, FIG. 15 shows the distal region along the longitudinal length of the ETT 1300, containing the cuff 1510, which overlies the shell 1520 and expandable frame 1530. A maximal inner radial force is exerted on the frame when the cuff 1510 is inflated (F__{c}), with the shell resisting further expansion (F_ₕ_ₑ), and the radial inward force (F__{Pi}) when patient inspires air. Hence the frame resistance force F__{RO}_ₑ should be designed equal to or greater than the sum of F__{c}, F_ₕ_ₑ and F__{Pi}.

### IV. Cuff Design

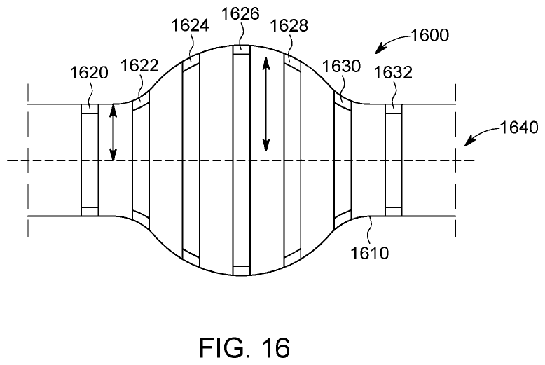

The above-described embodiments employ a collapsible cuff sealed, or otherwise secured, to the shell that inflates radially outwardly away from the substantially cylindrical surface of the expanded shell and frame. FIG. 16 details a cross section of the distal region of an ETT 1600 according to an alternate embodiment. The expandable shell 1610 is sized and arranged to overlie a series of frame hoops that, in an expanded configuration (as shown), increase in diameter from the cylindrical proximal tube shape (frame hoop 1620) through a series of longitudinally disposed, increasing and decreasing diameter, hoops 1622, 1624, 1628 and 1630 that define a cuff-like shape. The spring force of the shape and be designed to provide an appropriate seal against the airway, allowing air between the lungs and distal ETT connection/respiratory equipment to be isolated. Air flow from the ETT distal end 1640 via one or more tube-diameter hoops 1632. Note that the ETT 1600 can include a mechanism to collapse the cuff hoops, when removal of the ETT from the airway is desired. For example, a second retaining string can be used to unlatch a locking structure in the region of the cuff hoops, or otherwise actuate a collapse mechanism that should be clear to those of skill.

In both this embodiment, and the force-balanced embodiments described above, the cuff inflation/deployment avoids a reduction in the shell diameter that can undesirably constrict airflow.

### V. Detailed Embodiment

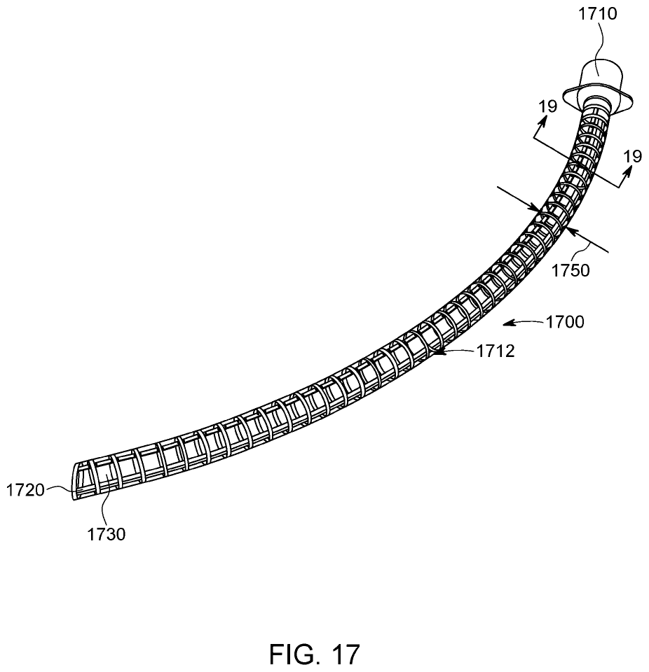

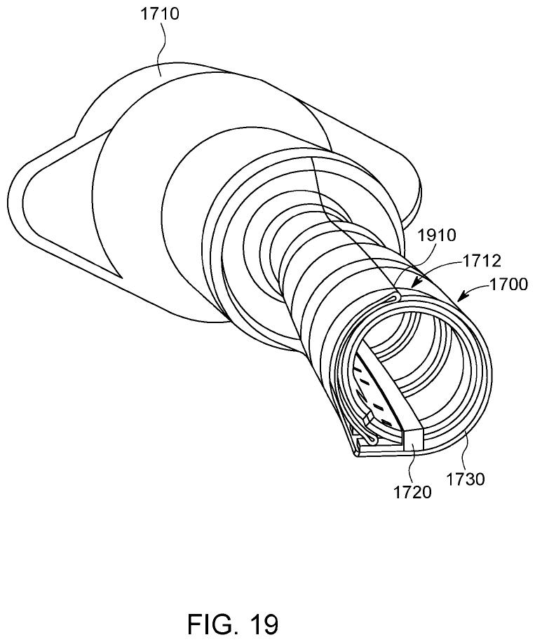

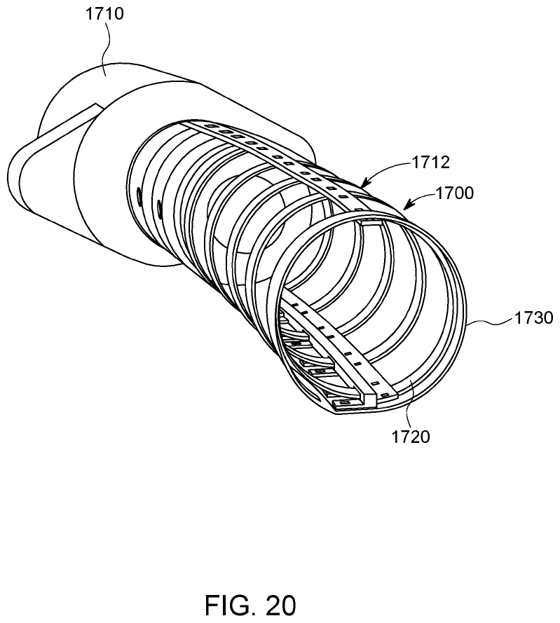

With reference to FIGS. 17-20, a full-length version of an illustrative embodiment of the ETT 1700 is shown. The elongated body 1712 is shown attached to a proximal adaptor 1710. In FIGS. 17 and 19 the frame 1720 is shown fully compressed (and retained by a string, etc. in a manner described above), such that the outer diameter 1750 of the body 1712 is reduced to a more readily insertable size. As shown more clearly in the cross section of FIG. 19, the shell defines a series 1730 of folds 1910 that hug the compressed frame 1720.

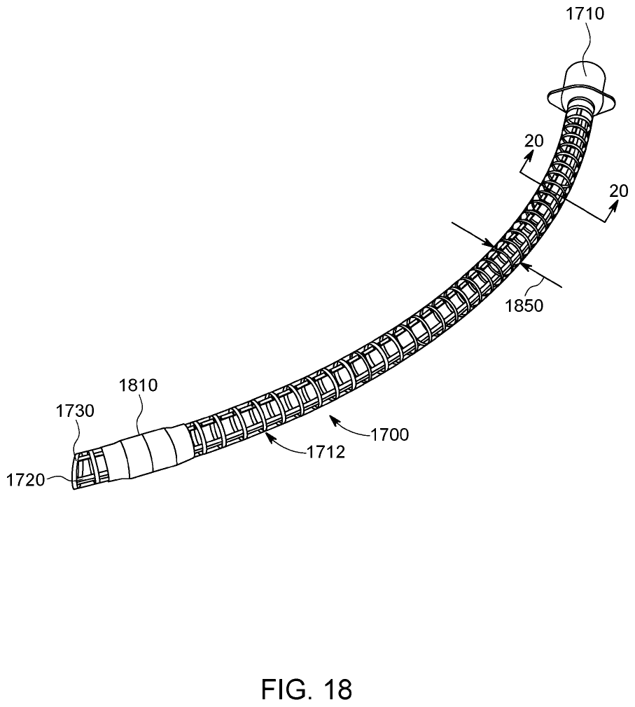

In the expanded state of FIGS. 18 and 20, the folds (1910) of the shell 1730 are removed by full expansion of the frame 1720, to a maximum outer diameter 1850, under the frame's internal hoop stress. The shell 1730 then acts as a retaining structure to further expansion of the frame 1720 as the overall structure achieves static equilibrium. As shown particularly in FIG. 18, the expanded body 1712 includes a distal cuff 1810 that defines an expandable layer, sealed to outer surface of the shell 1730, and which is inflatable via a pressure lumen (not show, described generally above). Note that a variety of cuff arrangements can be employed, as previously described.

### VI. Conclusion

It should be evident that the above-described ETT effectively reduces the tube's overall diameter while it is inserted the patient's airway, by selectively allow for a radial change in size. More particularly, the design allows the ETT to be delivered into the patient's airway with an outer diameter of approximately 5 mm, thereby facilitating insertion into even difficult airways, and then allows expansion to functioning volume when inside the patient's airway. When compressed, the compressed structure still enables a slip-fit over a 2.5 mm flexible bronchoscope so that clinicians or operators can continue utilizing these devices' effective mobility and visualization capabilities to deliver the expandable ETT. Advantageously, the expandable ETT of the various embodiments contemplated herein, by reducing the size of the product being inserted, reduces the need for general anesthesia, reduces the number of procedural challenges, and results in better patient outcomes.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein various directional and dispositional terms such as “vertical”, “horizontal”, “up”, “down”, “bottom”, “top”, “side”, “front”, “rear”, “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances of the system (e.g. 1-5 percent). Also, while a series of hoops disposed at relatively equal spacing from each other about their circumference are provided, the hoops can have variable width overall with respect to other hoops. The hoops can also have varying width at different points about their respective circumference and/or can be tilted along a plane perpendicular to the tube central axis. The spacing of hoops from adjacent hoops can also be variable along the longitudinal length of the ETT. Such varying width, spacing and/or tilt can occur at various locations along the longitudinal length to impart various physical properties to the structure—for example stronger or weaker spring force at different locations, or more less bending flexibility along the length. Accordingly, this description is meant to be taken only by way of example, and not to otherwise limit the scope of this invention.

What is claimed is:

1. An expandable endotracheal tube (ETT) comprising:
a frame having a plurality of hoops joined by longitudinal ribs, the hoops formed into a compressed configuration with torsional spring force imparted thereby;
a collapsible shell covering the frame that defines a sealed tube between a proximal end and a distal end; and
a retaining structure that maintains the hoops in the compressed configuration with the frame and the shell defining a compressed outer diameter, the retaining structure being selectively releasable to cause to the frame and the shell to expand to an expanded diameter that is greater than the compressed diameter.

2. The expandable ETT as set forth in claim 1, wherein the retaining structure comprises a member that passes through a plurality of holes located along overlapping circumferential positions of the frame and the shell folded therearound.

3. The expandable ETT as set forth in claim 2, wherein the retaining structure comprises a string under tension.

4. The expandable ETT as set forth in claim 3, wherein the string passes through holes in each of the ribs when adjacent to each other, the ribs moving circumferentially to circumferentially spaced-apart positions in the expanded configuration.

5. The expandable ETT as set forth in claim 4, wherein the frame includes a stop that resists compression after the frame expands to the expanded configuration.

6. The expandable ETT as set forth in claim 2, further comprising a cuff, adjacent to the distal end, that collapses in the compression, the cuff being interconnected to an inflation source having a conduit that extends along a longitudinal length of the frame and shell.

7. The expandable ETT as set forth in claim 2, wherein the frame defines an inner lumen in the compressed configuration adapted to slidably receive a flexible bronchoscope, or other delivery device.

8. The expandable ETT as set forth in claim 2, wherein the frame and shell are constructed and arranged to expand from a compressed outer diameter of approximately 5 mm to an expanded outer diameter of between approximately 8 mm and 10 mm.

9. The expandable ETT as set forth in claim 2, wherein the proximal end includes a fitting for connection to respiratory equipment.

10. The expandable ETT as set forth in claim 2, wherein the retaining structure comprises a semi-rigid rod.

11. The expandable ETT as set forth in claim 2, wherein the frame and shell are constructed and arranged to expand from a compressed outer diameter of between approximately 5 mm and 8 mm to an expanded outer diameter of between approximately 12 mm and 13 mm.

12. The expandable ETT as set forth in claim 1, further comprising;
another frame having a plurality of hoops joined by longitudinal ribs, the hoops formed into a compressed configuration with torsional spring force imparted thereby,
another collapsible shell covering the frame that defines a sealed tube between a proximal end and a distal end, and
a retaining structure that causes the other frame and the other shell to define a compressed outer diameter, and being releasable to cause to the frame and the shell to expand to an expanded diameter that is greater than the compressed diameter,
wherein the shell and the other shell are maintained adjacent to each other along at least a portion of respective lengths thereof to define a double lumen ETT.

13. The expandable ETT as set forth in claim 1, wherein at least a portion of the frame and the shell, adjacent to a distal end of the ETT defines, when in the expanded diameter, a cuff, adjacent to the distal end with a larger diameter than the adjacent shell at the expanded diameter so as to define a seal with an airway.

14. The expandable ETT as set forth in claim 13, further comprising a lumen for inflating the cuff to the large diameter based upon a proximally located inflation mechanism.

15. The expandable ETT as set forth in claim 13, further comprising a compression mechanism that is arranged to allow for returning the frame to a diameter that is smaller than the expanded diameter to facilitate removal from the airway

16. A method for using the expandable ETT as set forth in claim 2, comprising the steps of:
associating the ETT in the compressed configuration with a tracheal delivery device;
guiding the ETT into an airway of the patient using information from the delivery device;
disengaging the delivery device with respect to the ETT; and
actuating the retaining structure to place the ETT into the expanded configuration.

17. The method as set forth in claim 12 wherein the delivery device is a flexible bronchoscope that is selectively inserted within the lumen in the compressed configuration to assist guidance into the airway based upon visual feedback, and that is withdrawn therefrom when guidance is completed

18. The method as set forth in claim 11, further comprising, inflating a cuff adjacent to the distal end of the ETT to seal the airway.
